(19) United States
(12) Patent Application Publication
(10) Pub. No.: US 2025/0264480 A1
(43) Pub. Date: Aug. 21, 2025

(54) ASSAY METHODS, ASSAY CARTRIDGES, AND ASSAY DEVICES INCLUDING MAGNETIC PARTICLE DE-AGGREGATION

(71) Applicant: Siemens Healthineers Nederland B.V., Den Haag, Zuid-Holland (NL)

(72) Inventors: Aaron Kauffmann, Elkhart, IN (US); Jeffrey Mayfield, South Bend, IN (US); Alexander van Reenen, Vught (NL)

(21) Appl. No.: 18/859,161

(22) PCT Filed: Apr. 21, 2023
(86) PCT No.: PCT/EP2023/060412

§ 371 Date: Oct. 22, 2024

Related U.S. Application Data
(60) Provisional application No. 63/363,513, filed on Apr. 25, 2022.

(51) Int. Cl. G01N 33/68; G01N 33/543; G01N 33/553

(52) U.S. Cl. CPC G01N 33/6893; G01N 33/54386; G01N 33/553; G01N 2800/324

(57) ABSTRACT

An assay cartridge enabling improved measurement sensitivity of a target component (Troponin I or T) in the presence of high amount of albumin in a test sample. The assay cartridge includes a cartridge body including a sample inlet adapted to receive a patient sample and a reaction chamber coupled to the sample inlet, magnetic particles contained in the reaction chamber comprising a first antibody with a specific affinity towards the target component, electrodes provided at a location configured to be in contact with a rehydrated test sample containing the magnetic particles in the reaction chamber, and a sensor surface of the reaction chamber comprising a second antibody with a specific affinity towards the target component. Methods of quantifying a target component (e.g., a Troponin component) in immunoassay testing are provided, as are assay devices adapted to quantify a target component (e.g., Troponin I or T).

### FIELD

The present disclosure relates to methods, assay cartridges, and assay devices configured to measure a target component (e.g., a Troponin component) in a test sample.

### BACKGROUND

An immunoassay that is configured to measure cardiac Troponin (e.g., Troponin I or Troponin T) can provide a tool for diagnosing myocardial infarction, for example. While most contemporary assays provide adequate diagnostic performance, increased sensitivity of high sensitivity assays introduced into clinical practice provide the potential to further shorten intervals between blood draws and/or the time needed to detect significant Troponin elevations. Furthermore, high sensitivity cardiac Troponin assays offer improvements for predicting major adverse cardiovascular events. Such high sensitivity assays can measure Troponin concentrations in 50%=−100% of healthy individuals and therefore allow for the distribution of Troponin values within a healthy cohort to be measured, patient's baseline troponin levels to be monitored, and clinicians to be alerted to deteriorating cardiac conditions. These high sensitivity assays may aid in predicting a patient's risk of future adverse events and for guiding and monitoring corresponding adjustments to therapeutic interventions. Such high-sensitivity cardiac Troponin I assays, for example, can detect levels above 14 ng/l, which can signal the likelihood of heart damage or a heart attack.

In some cases, a condition of the test sample may reduce the sensitivity of such high sensitivity Troponin assays. This disclosure is directed at assay methods, biosensor apparatus (assay cartridges), and assay devices that may increase the sensitivity of such assays configured to measure a target component, such as a cardiac Troponin component, when such conditions are encountered.

### SUMMARY

This disclosure is directed, in one aspect, to a biosensor configured as an assay cartridge that is capable of measuring a target component, such as a Troponin component.

In another embodiment, a method of quantifying a target component (e.g., a Troponin component such as Troponin I or Troponin T, for example) in a test sample is provided. The method of quantifying the target component comprises providing a reaction chamber containing magnetic particles including a first antibody and a sensor surface including a second antibody, flowing the test sample into the reaction chamber, rehydrating the magnetic particles and binding a target component to the first antibody of the magnetic particles, and after rehydration, exposing the magnetic particles to a pulsed voltage whereby clumping of the magnetic particles in the presence of albumin in the test sample is reduced.

In an apparatus embodiment, an assay cartridge adapted for measuring a target component (e.g., a Troponin component) in immunoassay testing is provided. The assay cartridge comprises a cartridge body including a sample inlet adapted to receive a patient sample and a reaction chamber coupled to the sample inlet, magnetic particles contained in the reaction chamber comprising a first antibody with a specific affinity towards the target component, electrodes provided at a location configured to be in contact with a rehydrated test sample containing the magnetic particles in the reaction chamber, and a sensor surface of the reaction chamber comprising a second antibody with a specific affinity towards the target component.

In another apparatus embodiment, an assay device configured to improve sensitivity of immunoassay testing of a target component (e.g., Troponin I or Troponin T) is provided. The assay device comprises a device body including a cartridge receiver, an assay cartridge received in the cartridge receiver, the assay cartridge comprising a cartridge body including an inlet channel adapted to receive a test sample and a reaction chamber therein, magnetic particles having a first antibody with a specific affinity towards the target component provided in the reaction chamber, electrodes provided at locations configured to be in contact with a rehydrated test sample containing the magnetic particles in the reaction chamber, and a voltage pulse generator coupled to the electrodes and configured to provide one or more voltage pulses to the electrodes.

Still other aspects, features, and advantages of this disclosure may be readily apparent from the following description and illustration of a number of example embodiments, including the best mode contemplated for carrying out the disclosure. This disclosure may also be capable of other and different embodiments, and its several details may be modified in various respects, all without departing from the scope of the disclosure. It is to be understood that embodiments of the methods described herein are capable of operation in other sequences than described or illustrated herein. This disclosure is intended to cover all modifications, equivalents, and alternatives falling within the scope of the claims.

### BRIEF DESCRIPTION OF THE DRAWINGS

The drawings, described below, are for illustrative purposes, may be exaggerated in size, and thus are not necessarily drawn to scale. Any reference signs in the claims shall not be construed as limiting the claim scope. Accordingly, the drawings and descriptions thereof are to be regarded as illustrative in nature, and not as restrictive. The drawings are not intended to limit the scope of the disclosure in any way.

FIG. 1A illustrates a top schematic view of a first embodiment of an assay cartridge installed in an assay device according to one or more embodiments of the disclosure.

FIG. 1B illustrates a cross-sectioned end view of a first embodiment of an assay cartridge installed in an assay device taken along section 1B-1B of FIG. 1A according to one or more embodiments of the disclosure.

FIG. 1C illustrates another cross-sectioned end view of a first embodiment of an assay cartridge installed in an assay device taken along section 1C-1C of FIG. 1A according to one or more embodiments of the disclosure.

FIG. 1D illustrates a cross-sectioned side view of a first embodiment of an assay cartridge installed in an assay device taken along section 1D-1D of FIG. 1A according to one or more embodiments of the disclosure.

FIG. 1E illustrates an enlarged cross-sectioned end view taken through a reaction chamber of an assay cartridge illustrating re-hydration and clumping of magnetic particles in a test sample according to one or more embodiments of the disclosure.

FIG. 1F illustrates an enlarged cross-sectioned end view taken through the reaction chamber of an assay cartridge illustrating breaking up of the clumping of magnetic particles after application of pulsed voltage across the reaction chamber according to one or more embodiments of the disclosure.

FIG. 1G illustrates an enlarged cross-sectioned end view taken through the reaction chamber of an assay cartridge illustrating separation of unbound magnetic particles from magnetic particles having a target component complexed to a sensor surface after application of a magnetic field to the reaction chamber according to one or more embodiments of the disclosure.

FIG. 1H illustrates an enlarged partial cross-sectioned end view taken through a reaction chamber of an assay cartridge, prior to insertion of the test sample, functionally and structurally depicting one embodiment including magnetic particles having a first antibody affixed to a surface of the reaction chamber and a sensor surface including a second antibody bound thereto according to one or more embodiments of the disclosure.

FIG. 1I illustrates an enlarged partial cross-sectioned end view taken through a reaction chamber of an assay cartridge after insertion of the test sample including a target component to be quantified, functionally and structurally depicting an embodiment where magnetic particles are clumped in the presence of a high amount of albumin according to one or more embodiments of the disclosure.

FIG. 1J illustrates an enlarged partial cross-sectioned end view taken through a reaction chamber of an assay cartridge after application of a pulsed voltage, functionally and structurally depicting an embodiment where the magnetic particle clumping in the presence of a high amount of albumin is reduced (eliminated entirely as shown) according to one or more embodiments of the disclosure.

FIG. 1K illustrates an enlarged partial cross-sectioned end view taken through a reaction chamber of an assay cartridge after application of a magnetic field to the reaction chamber, functionally and structurally depicting an embodiment where magnetic particles having the target component bound thereto being moved in order to complex to a second antibody provided on a sensor surface according to one or more embodiments of the disclosure.

FIG. 2A illustrates a schematic top view of a second embodiment of an assay cartridge including electrodes, which when energized, are adapted to break up magnetic particle clumping and provide improved quantification of a target component in a test sample according to one or more embodiments of the disclosure.

FIG. 2B illustrates a voltage pulse generator circuit that can be coupled to an assay cartridge including electrodes according to one or more embodiments of the disclosure.

FIGS. 3A and 3B illustrate pictorial results (at 50X) of particle counts before (FIG. 3A—control) and after (FIG. 3B) application of a pulsed voltage to a test sample including a high volume of albumin therein illustrating the higher particle count as well as improved, more homogenous, particle distribution according to one or more embodiments of the disclosure.

FIG. 4 illustrates a flow chart of a method of quantifying a target component (e.g., Troponin I or Troponin T) in a test sample according to one or more embodiments of the disclosure.

### DETAILED DESCRIPTION

This disclosure is directed at improving the sensitivity of assays, such as cardiac Troponin assays, where the patient sample contains a high level of albumin therein. In one aspect of the disclosure, it was discovered by the inventors that high levels of albumin contained in the patient sample can affect the sensitivity of the assay (e.g., a Troponin assay) conducted using a test sample derived from the patient sample. Normal albumin levels range from 3.4 to 5.4 g/dL in whole blood. When the albumin level is high (i.e., provided in an amount greater than 5.4 g/dL in whole blood), clumping and agglomeration of the magnetic label (e.g., magnetic particles) utilized in the Troponin assay for binding the target component (e.g., Troponin I or Troponin T, for example) can occur. Where albumin is present in the patient sample 105 in a quantity greater than 10 g/dL even greater extent of clumping can occur, and especially when albumin is present in the patient sample 105 in a quantity greater than 100 g/dL in whole blood. This clumping can result in a lesser portion of the respectively clumped magnetic particles being available for complexing the target component (e.g., Troponin I or Troponin T) to a sensor surface thus reducing the sensitivity of target component detection.

In one or more embodiments, clumping in the assay (e.g., a Troponin assay) can be minimized by exposing the test sample (e.g., plasma) that has rehydrated magnetic particles therein to one or more pulses of an electric voltage potential (V). In some embodiments, a reaction chamber containing magnetic particles that have been rehydrated within plasma derived from the patient sample (e.g., whole blood) are exposed to the voltage potential (V). In some embodiments, the reaction chamber containing magnetic particles that have been rehydrated within the test sample (e.g., plasma) is subjected to successive voltage pulses, such as two or more pulses of electrical voltage potential (V) to facilitate breaking up of clumps of magnetic particles that can occur when a high amount of albumin is present in the patient sample.

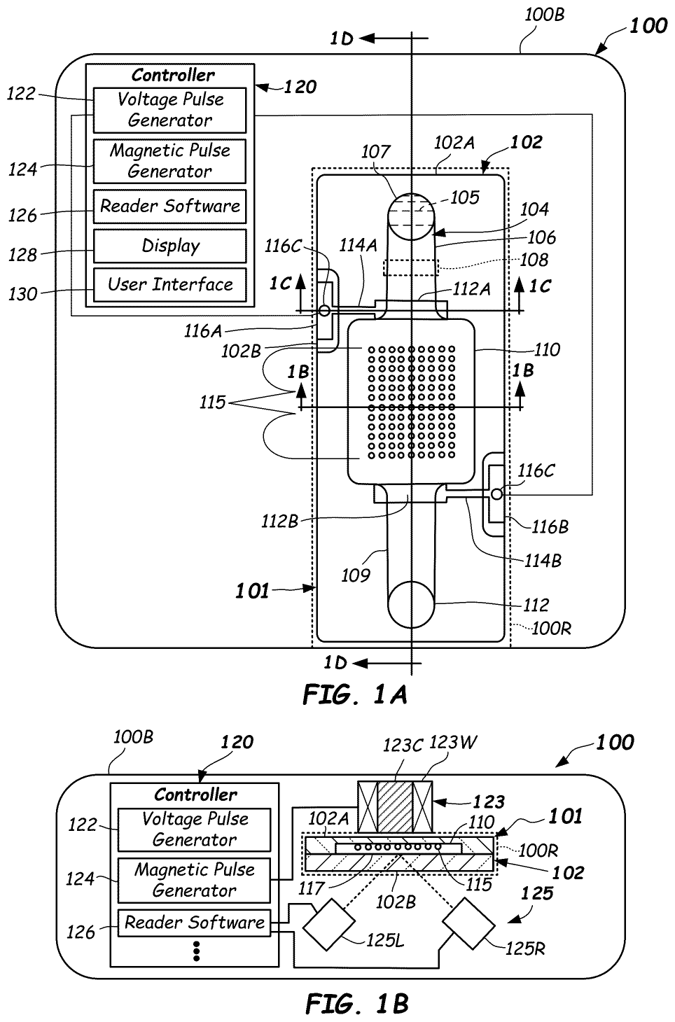

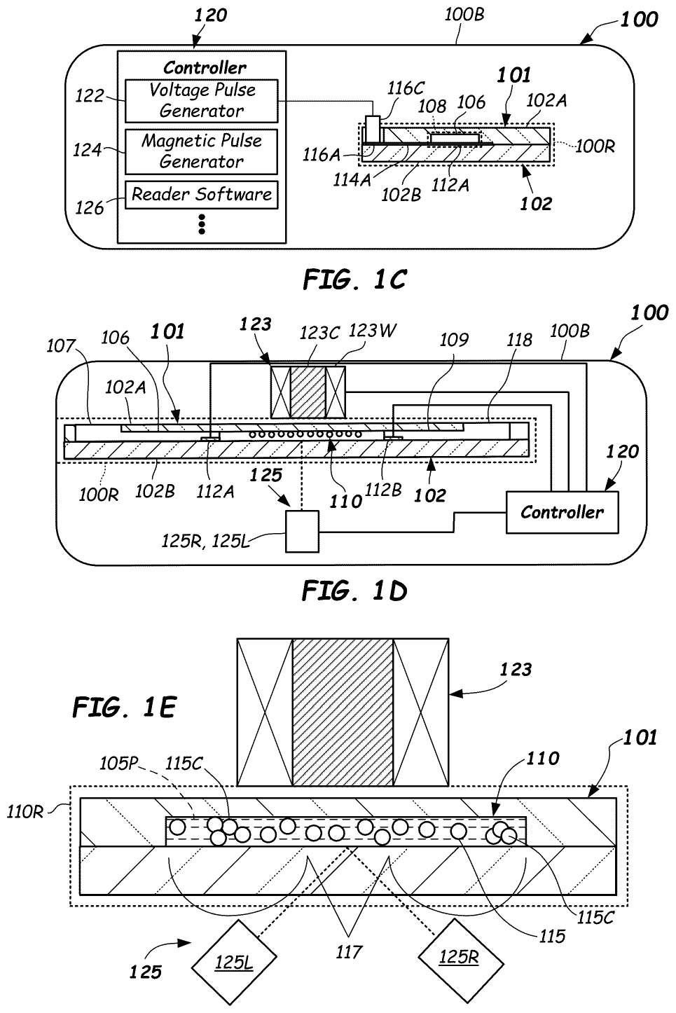

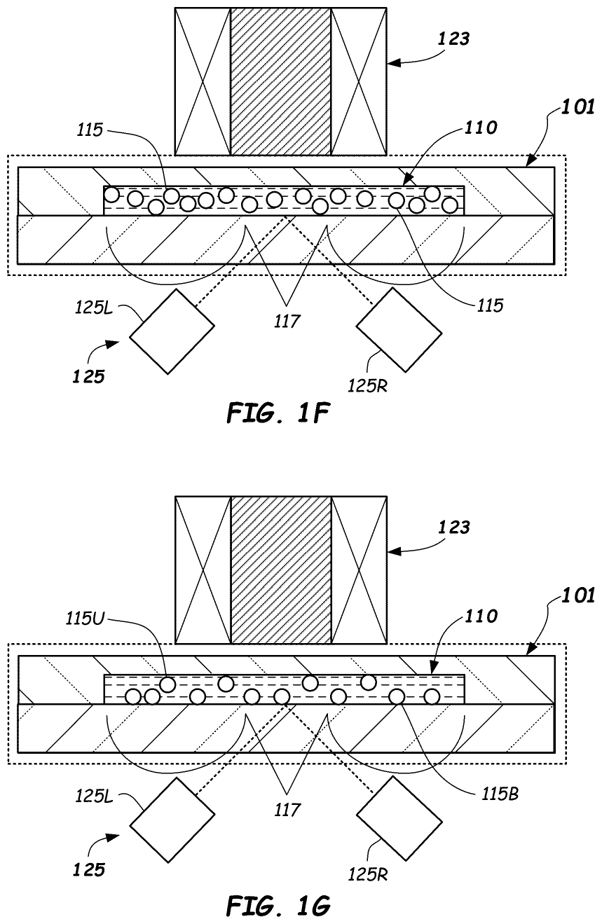

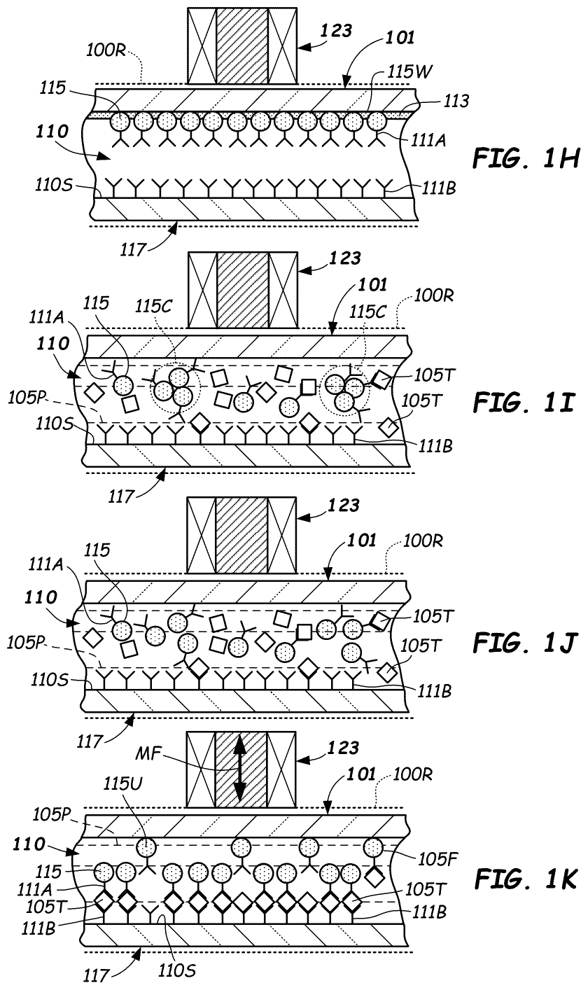

Further details of the method enabling improved sensitivity of such assays (e.g., Troponin assays) when the patient sample contains high levels of albumin are described with reference to FIGS. 1A-4 herein. Assay cartridges enabling improved measurement sensitivity of the target component (e.g., Troponin I or Troponin T) in the presence of such high albumin levels in the patient sample are also described herein, as well as assay devices utilizing such assay cartridges.

### Definitions

The following definitions are provided solely to aid in the Understanding of this disclosure. These definitions should not be construed to have a scope less than understood by a person of ordinary skill in the art.

“Assay” as used herein means an analysis or examination to determine a presence and/or quantity of a target component of a substance, such as a biological fluid sample from a patient.

“Assay cartridge” as used herein means a cartridge structure that is receivable in a receiver of an assay device.

“Assay device” as used herein means an apparatus configured to carry out an assay, such as a point-of-care instrument adapted to test for a Troponin component.

“Target component” as used herein means a specific component sought to be quantified that is contained in a test sample to be analyzed.

“Troponin I and Troponin T” are subunit of the troponin complex (Tn) that is used to diagnose cardiovascular disease and myocardial infarction.

“Sensor surface” as used herein refers to a surface to which antibodies are coupled and that allows the detection of magnetic particles complexed to such surface.

“Rehydration” as used herein means the constituents of the assay, such as magnetic particles and dry reagents, have been assimilated into the test sample as a rehydrated test sample.

“Clump” as used herein means a plurality of particles that are adhered to one another and that are coalesced into an agglomerated particle mass.

FIGS. 1A-1D schematically illustrates a first embodiment of a biosensor configured as an assay cartridge 101 received in an assay device 100. The assay cartridge 101 includes a body 102 of suitable material providing a configuration including at least one sample inlet 104 that can be configured to receive a patient sample 105 of biological material that has been obtained from a patient via a pinprick or otherwise. The patient sample 105 may be whole blood and may be deposited into a well 107 formed in the body 102. An inlet channel 106 formed in the body 102 provides a flow passageway for the patient sample 105 to flow from the sample inlet 104 towards a reaction chamber 110.

In the depicted embodiment, a filter 108 can be provided in the inlet channel 106 between the sample inlet 104 and the reaction chamber 110, or even within the well 107 in some embodiments. The filter 108, when the patient sample 105 is whole blood, operates to remove cellular components such as red blood cells and other solid portions of the whole blood and leaves only the plasma as a test sample 105P to flow into the reaction chamber 110. Thus, the inlet channel 106 is coupled to the reaction chamber 110 and is configured to flow the test sample 105P into the reaction chamber 110, wherein the inlet channel 106 can be formed as a capillary channel.

However, in some embodiments, the whole blood may be separated into cellular components and plasma by centrifugation and only the plasma alone may be deposited directly into the well 107 as the test sample 105P. In this embodiment where the test sample 105P is plasma deposited into the well 107, the filter 108 is not needed, and would not be present. In this embodiment, the test sample 105P may flow through the inlet channel 106 to the reaction channel 110 via capillary action.

Again referring to FIGS. 1A-1D, the assay device 100, comprises a device body 100B, such as a molded plastic body, including a cartridge receiver 100R that is configured to receive the assay cartridge 101 therein. The cartridge receiver 100R can be a port or other opening into which the assay cartridge 101 can be inserted or placed. In some embodiments, the cartridge receiver 100R can include a door (not shown) that is opened to allow the insertion of the cartridge 101 after which the door may be closed.

The assay cartridge 101 includes the cartridge body 102. The cartridge body 102 may be made of two or more body parts of material that can be adhered or otherwise bonded or fastened together. The connection can be by any suitable mechanism, such as through the use of an adhesive. For example, one body part, such as first body part 102A may include recessed portions formed therein to aid in construction of the inlet channel 106, reaction chamber 110, as well as an outlet channel 109. The well 107 may be formed as a hole passing fully through the first body part 102A and its interaction with a surface of the second body part 102B. The first body part 102A and second body part 102B can cooperate and engage one another to collectively form the well 107, sample inlet 104, inlet channel 106, reaction chamber 110, outlet channel 109, and outlet 118. The outlet 118 may also be formed as a hole passing fully through the first body part 102A and its interaction with a surface of the second body part 102B. Optionally, a vent may be provided that is connected to the outlet channel 109. Other suitable configurations can be used to form the reaction chamber 110 and inlet channel 106, such as including recesses only in the second body part 102B, or in both of the first body part 102A and the second body part 102B.

In the depicted embodiment, the second body part 102B may be highly transparent and may be a planar body at least in the area of the reaction chamber 110. The first body part 102A may be transparent or translucent, or even opaque in some embodiments, for example. The first body part 102A and second body part 102B may be made of glass, plastic, or other suitable moldable or formable materials. The second body part 102B should be as optically transparent as possible when optical methods are used to quantify the target component 105T after the assay reaction is completed, such as transparent glass, acrylic, polystyrene, styrene-acrylonitrile, polycarbonate, polyethylene terephthalate, and combinations thereof.

By way of example, the reaction chamber 110 may be about 1.5 mm long×1.0 mm wide and 0.25 mm in height and may include dried reagent therein to be described later herein. The inlet channel 106 and the outlet channel 109 may be appropriately sized to facilitate liquid capillary action. For example, the inlet channel 106 and outlet channel 109 may include cross-sectional area of about 0.125 mm², for example. Other suitable channel and reaction chamber sizes may be used.

In order to facilitate quantification of the target component 105T present in the test sample 105P, magnetic particles 115 can be utilized as a target component binding agent. The target component 105T may be a Troponin component, such as Troponin I (cTnI), although other target components, such as Troponin T (cTnT) may instead be quantified. Other target components that are cardiac markers may also be quantified. Other target components may benefit from the anti-aggregating properties achieved by the embodiments described herein

The magnetic particles 115 can have attached thereto, such as through a suitable coating or application process, a first antibody 111A with a specific affinity towards binding to the target component 105T (e.g., Troponin I or Troponin T). Also provided in the reaction chamber 110 is a second antibody 111B that can be coupled to a sensor surface 110S of the reaction chamber 110. The sensor surface 110S may be provided as part of a quantification window 117 through which detection and quantification of the target component 105T may be accomplished. The sensor surface 110S can be located proximate to the quantification window 117 of the reaction chamber 110, such as on one side thereof and may be approximately the same size as the quantification window 117.

The sensor surface 110S can be a specially derivatized surface to which molecules, more particularly antibodies (or functional fragments thereof) can be bound. Examples of suitable sensor surfaces 110S can include, glass, plastic, an organic crystal or an inorganic crystal (e.g., silicon), an amorphous organic material, or an amorphous inorganic material (e. g. silicon nitride, silicon oxide, silicon oxynitride, aluminum oxynitride, aluminum oxide). Other suitable surfaces can be used.

In some embodiments, the quantification of the target component 105T can be performed optically. Such optical detection can be performed by detecting a quantity of magnetic particles 115 that are complexed to the sensor surface 110S, such as by frustrated total internal reflection (FTIR). In yet other embodiments, the magnetic particles 115 complexed to the sensor surface 110S can be detected by other optical means.

In some embodiments, the first antibody 111A can be a monoclonal antibody, such as a monoclonal anti-troponin I antibody. Optionally, the first antibody 111A can be a monoclonal antibody, such as a monoclonal anti-troponin T antibody. The monoclonal antibody according to the present disclosure can also include Fab fragments from the monoclonal antibody, aptamers, affibodies, scFv fragments, and any other single epitope binding moiety known to the person of ordinary skill in the art.

In some embodiments, monoclonal antibodies 111A are coupled to the magnetic particles 115 so as to allow manipulation of the labeled antibody via a suitable magnetic field generator provided in the assay device 100 that is operable with the assay cassette 101. For example, the assay device 100 may include a magnetic field generator 123, which may comprise any suitable form of an electromagnet or moveable magnet. Magnetic field generator 123 may include a core 123C and a winding 123W of sufficient size and number of winds to generate, when energized via a signal from a magnetic pulse generator 124, a sufficient magnetic field MF to allow manipulation and movement of the magnetic particles 115 through the test sample 105P. Such magnetic particles may be manipulated as described in U.S. Pat. No. 9,720,003, for example, to maximize forming of complexes to the sensor surface 110S.

In some embodiments, the second antibody 111B can be a polyclonal antibody such as a polyclonal anti-Troponin I antibody and can be coupled to the sensor surface 110S. For example, goat polyclonal anti-Troponin I antibody was found to be quite effective. The use of goat polyclonal antibodies can lead to optimized assay results. The polyclonal antibody according to the present disclosure can also include Fab fragments from polyclonal antibodies, and any group of binding moieties with variable structure known to the person of ordinary skill in the art.

In some embodiments, the antibodies according to the present disclosure are directed at amino acid sequence 30-110, or even 80-110, of the Troponin I molecule. This is a generally stable part of the molecule. Preferably the epitopes chosen do not overlap with known regions for heparin binding, which may result in interference from heparinized patient samples.

In some embodiments, the monoclonal anti-Troponin I antibody can be selected from the group comprising clones: a34500 (binding to amino acid 87-91 of Troponin I), 81-7 (a34780 (binding to amino acid 136-154 of Troponin I), a34650 (binding to amino acid 41-49 of Troponin I), 267 (ab 14530, binding to amino-acid 169-184 of Troponin I), 16A11 (a24460, binding to amino-acid 87-91 of Troponin I), 19C7 (a19615, binding to amino-acid 41-49 of Troponin I), 560 (binding to amino acid 83-93 of cTnI), or combinations thereof. The use of these clones can lead to improved selectivity in the magnetic immunoassay according to the disclosure.

Certain procedures can be used to provide the antibodies on the magnetic particles that are used in the Troponin quantification assay. For example, an Ademtech protocol, known to a person of ordinary skill in the art can be used to coat the magnetic particles 115 with the first antibody 111A. In this protocol, monoclonal antibody (e.g., first antibody 111A) at a concentration of, for example, 20 μg antibody per mg of magnetic particle 115 can be coupled to carboxylated magnetic particles in the presence of EDC (1-Ethyl-3-(3-dimethylaminopropyl)-carbodiimide). Other suitable methods for coupling the first antibody 111A comprising the monoclonal antibody to the magnetic particles 115 may be used.

The magnetic particles 115, which are sometimes referred to as magnetic labels, can be provided in the reaction chamber 110 prior to receiving the test sample 105T therein. For example, the magnetic particles 115 may be provided on one or more walls 110W of the reaction chamber 110, such as shown in FIG. 1H. The magnetic particles 115 may be adhered to a wall by any suitable binding material 113 in some embodiments, such as by any material that is non-interfering with the assay. For example, the binding material 113 may be a sugar and protein solution that includes the magnetic particles 115, and which is applied to the wall of the reaction chamber 110 and then dried. Other suitable binding materials may be used.

In addition to the magnetic particles 115, the dry reagents can be provided at a suitable location in the reaction chamber 110. The dry reagents include the buffer components necessary for conducting the assay. The components of the dry reagents can be deposited and dried individuality at different locations in the reaction chamber 110 or together at the same location. The dry reagents can be deposited via several drying techniques including lyophilization, for example. Lyophilization prevents the formation of crystals and allows the reagents to be dried to an amorphous glassy state that is readily re-dispersed and rehydrated upon the addition of the test sample 105P (e.g., plasma) into the reaction chamber 110.

The sensor surface 110S as shown in FIG. 1H-1K according to some embodiments of the present disclosure can be functionalized with the second antibody 111B, which can comprise the polyclonal antibody. Polyclonal antibodies are preferred as the degrees of freedom for binding of Troponin I, especially when bound to the monoclonal anti-Troponin I antibody of the magnetic particles 115 are limited. One advantage is a consequence of the variability in orientation of polyclonal antibodies immobilized on a sensor surface 110S due to the different antibody types of a polyclonal. Further advantages can arise by increasing the amount of suitable binding sites by using polyclonal antibodies directed to different epitopes leading to optimized assay results. Due to favorable assay sensitivity, polyclonal goat antibodies specific for Troponin I or Troponin T can be used.

In another embodiment, an additional antibody specific for Troponin I, or a mixture of several antibodies specific for Troponin I can be coated on the sensor surface 110S. Preferably the additional antibody is a monoclonal antibody. In another embodiment, the sensor surface 110S can be coated with at least two, even more preferably at least three, different monoclonal anti-Troponin I antibodies or a mixture of monoclonal troponin I antibodies. This also leads to formation of a “polyclonal” antibody on the sensor surface 110S as receptors, as binding sites for more than one epitope are present. This situation functionally resembles coating with a polyclonal antibody and thus is considered a polyclonal antibody herein.

The polyclonal antibody 111B can, for example, be coated onto the sensor surface 110S by any suitable process. For example, the second antibody 111B (e.g., polyclonal antibody) can be provided on the sensor surface 110S by inkjet printing a plurality of drops (e.g., of approx. 2 nL each) at a desired concentration (e.g., of about 150 μg/mL). Other suitable drop sizes and concentrations may be used. Printing antibody pattern features such as dots, squares, text, fine lines, or combinations thereof can be used. Optionally, the second antibody 111B may be bound to the sensor surface 110S by other processes such as microcontact printing, immersion coating (in a bulk solution), and/or drop coating (from a nanopipette or micropipette).

In more detail, the magnetic particles 115 may be attached to a wall 110W, such as to a wall located opposite from the sensor surface 110S. Optionally, the magnetic particles 115 may be attached to other walls, such as to the side walls of the reaction chamber 110 or even to the sensor surface 110S itself along with the second antibody 111B. Attachment may be by way of any suitable binding material 113, as previously discussed. In some embodiments, the magnetic particles 115 can have a size (approximate diameter) of about 200 nm to 1000 nm. Particles in this size range allow for optimal assay conditions and detection.

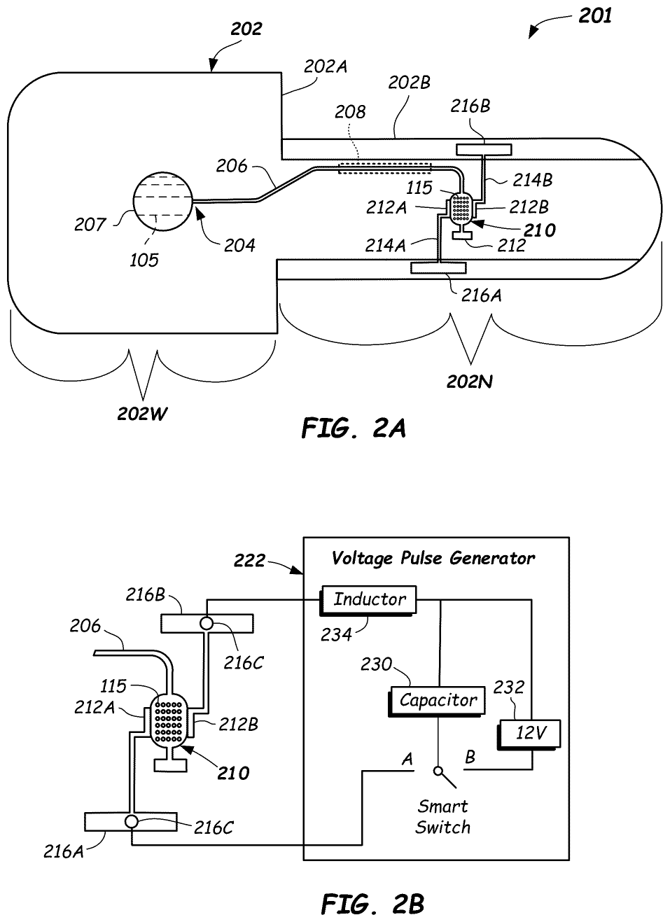

In more detail, the assay cartridge 101 includes electrodes 112A, 112B that are provided at locations configured to be in contact with the test sample 105P containing the magnetic particles 115 in the reaction chamber 110. This allows for a desired pulsed voltage to be applied to the test sample 105P containing the magnetic particles 115 in the reaction chamber 110. The electrodes 112A, 112B may be made from any suitable electrically conductive material, such as silver, copper, gold, or other conductive metals or materials. For example, the electrodes 112A, 112B may be provided on opposite sides of the reaction chamber 110, such as proximate the inlet channel 106 and outlet channel 109, as shown. Optionally, the electrodes 112A, 112B may be provided on other opposite sides of the reaction chamber 110, such as is shown in FIG. 2A.

The electrodes 112A, 112B may be connected to contact pads 116A, 116B that are exposed so to be able to be contacted by electrical contacts 116C (FIG. 1C) that are part of the assay device 100. Electrical contacts 116C may be pogo type or other suitable spring-loaded contacts, for example. The contact pads 116A, 116B may be interconnected to the electrodes 112A, 112B by conductor paths 114A, 114B. The electrodes 112A, 112B, conductor paths 114A, 114B, and contact pads 116A, 116B may be formed by any suitable method, such as by any coating, masked deposition or plating method, such as vapor deposition, laser-induced transfer deposition, electroless plating, immersion coating, sputtering, gilding, electroplating, and the like, or otherwise adhering a formed conductive member made up of the electrodes 112A, 112B, conductor paths 114A, 114B, and contact pads 116A, 116B, or combinations thereof.

A voltage pulse generator 122, which may be included as part of a controller 120, or otherwise provided, may be electrically coupled to the electrodes 112A, 112B through the contact pads 116A, 116B and conductor paths 114A, 114B. The voltage pulse generator 122 can be configured to provide one or more voltage pulses to the electrodes 112A, 112B. This exposes the test sample 105P containing rehydrated magnetic particles 115 and rehydrated reagents to one or more pulses of voltage potential V. Thus, clumping 115C is effectively reduced and may be minimized or even eliminated.

The applied voltage potential V between the respective electrodes 112A, 112B may have an initial magnitude of greater than 150 V in some embodiments, and an initial magnitude greater than 300 V in other embodiments. The pulsed voltage V may be applied once, and in some embodiments, more than once. The pulsed voltage magnitude may decay to some extent during its duration of exposure after its initial provision. For example, the exposing of the test sample 105P including rehydrated magnetic particles 115 to the pulsed voltage V can comprise providing at least two voltage pulses in succession to break up clumping 115C of the magnetic particles 115 in the presence of albumin in the test sample 105P. For example, each pulse can have a time duration of greater than 2 milliseconds. In some embodiments where multiple pulses are successively applied, each of the at least two pulsed voltages can have a duration of greater than 2 milliseconds, but the magnitude of the voltage V may fall off (be reduced) somewhat during each duration. In some embodiments, the magnitude of the initial voltage pulse V may be different (e.g., lower) in successive pulses that are applied. Thus, each pulse may be of a different amplitude and/or duration. The voltage pulse should be low enough in magnitude and applied in a short enough duration so as to not cause cell lysis.

By way of example, and not by limitation, the voltage pulse generator 122 can include any suitable circuitry having the ability to generate a desired voltage pulse profile. For example, FIG. 2B illustrates one possible configuration of a voltage pulse generator 222 that can be used as the voltage pulse generator for the FIG. 1A-1D embodiment. This embodiment uses a smart switch that can be toggled between A and B as programmed to charge and discharge a capacitor 230 using a DC voltage source 232 such as a 12V battery, for example. An inductor 234 may be included to control the resonant frequency. Suitable values of capacitance can range from 25 μF to 1500 μF and from 2 mH to 9 mH for the inductor 234, for example. A designed resistance can be used in the circuit to provide damping to the pulses, such as critical damping.

FIG. 2A illustrates another embodiment of assay cartridge 201 that is configured to conduct an assay in an assay device 100, such as assay device 100 in order to quantify a target component 105T, such as a Troponin component (Troponin I or Troponin T). The assay cartridge 201 includes all the elements of the FIG. 1A-1D embodiment, except that the body 202 includes a key shape, having a wider width portion 202W and a narrower width portion 202N. The wider width portion 202W can have formed therein the well 207, inlet 204, and part of the inlet channel 206. The narrower width portion 202N can include the remainder of the inlet channel 206, the reaction chamber including the magnetic particles 115 and dry reagents therein. Further, the narrower width portion 202N can include a vent 212. The electrodes 212A, 212B, conductor paths 214A, 214B, and contact pads 216A, 216B may be provided on the first body part 202A by any suitable method, such as the methods described herein. The second body part 202B may include recesses therein forming, in combination with the first body portion, the well 207, inlet channel 206, reaction chamber 210, and vent 212. As before, a filter 208 may be included to allow only the test sample 105P (e.g., plasma) to flow into the reaction chamber 210 when whole blood is deposited in the well 207 as the patient sample 105. As is shown in FIG. 2B, the contact pads 216A, 216B may be connected to the voltage pulse generator 222 by any suitable electrical contacts 216C as described herein.

To better understand the various operations that are undertaken during the process of conducting the assay, the following is offered with reference to FIGS. 1H-1K. According to some embodiments, the assay uses two different antibodies, a first antibody 111A (e.g., a monoclonal anti-Troponin I or anti-Troponin T antibody) coupled to the magnetic particles 115 (the magnetic labels) and a second antibody 111B (e.g., a polyclonal anti-Troponin I or anti-Troponin T antibody) coupled to the sensor surface 110A proximate the quantification window 117. The location of the first antibody 111A and the second antibody 111B may be switched in some embodiments. For example, the first antibody 111A may be a polyclonal anti-Troponin I or anti-Troponin T antibody and may be coupled to the magnetic particles 115 (the magnetic labels), and the second antibody 111B may be a monoclonal anti-Troponin I or anti-Troponin T antibody that is coupled to the sensor surface 110S.

FIG. 1H shows a schematic depiction of a portion of the reaction chamber 110 embodied in an assay cartridge 101 according to embodiments of the disclosure. In FIG. 1I a test sample 105P potentially comprising the target component 105T (e.g., Troponin I or Troponin T) in the presence of albumin is introduced into the reaction chamber 110 and this operates to rehydrate the magnetic particles 115 and the dried reagents that are present in the reaction chamber 110. In the presence of high amounts of albumin, the magnetic particles 115 can aggregate and produce clumps 115C in the reaction chamber 110, which are unwanted.

FIG. 1J illustrates the application of the pulsed voltage V (e.g., one or more voltage pulses) and that the clumps 115C previously present in FIG. 1I are broken up and reduced, minimized, or even substantially eliminated. The exposure of the test sample 105T including the clumped magnetic particles 115C to the pulsed voltage V can involve one and preferably more than one successive pulse of a voltage potential V applied between the electrodes 112A, 112B. The simple presence of the magnetic particles 115 in the test sample 105P may facilitate coupling and binding of some of the target component 105T present in the test sample 105P to the magnetic particles 115 as shown schematically in FIG. 1J. In particular, the target component 105T, if present, can come into contact with the first antibody 111A and becomes coupled to the magnetic particles 115. The test sample 105P additionally comes into contact with the second antibody 111B coupled to a sensor surface 110S. The first antibody 111A and second antibody 111B are selected such that the analyte of interest (the target component 105T, e.g., Troponin I or Troponin T) can be bound to both the first antibody 111A and the second antibody 111B simultaneously, and thus form a complex on the sensor surface 110S as best shown in FIG. 1K. In particular, if the target component 105T is present in the sample 105P, complexes made up of the magnetic particle with first antibody 111A, the target component 105T, and the second antibody 111B will be formed on the sensor surface 110S.

Following the exposing the test sample 105P in the reaction chamber 110 to the pulsed voltage, the magnetic particles 115 can be exposed to various magnetic fields MF from a magnetic field generator 123 to assist in moving unbound magnetic particles 115U away from the sensor surface 110S and/or moving them towards the sensor surface 100S and also assisting in the formation of complexes by binding the target component 105T bound to the first antibody 111A to the second antibody 111B at a sensor surface 110S. In particular, to enhance and maximize the formation of such complexes, the magnetic field MF may be applied to the reaction chamber 110 as shown in FIG. 1K. The magnetic field MF may be reversed in flux direction one or more times to facilitate movement (at least translation) of the magnetic particles 115 within the test sample 105P towards and away from the sensor surface 110S so as to aid in forming a maximum number of complexes. This can involve alternating the direction of a magnetic field MF perpendicular to the sensor surface 110S as described in U.S. Pat. No. 9,720,003, or optionally parallel to the sensor surface 110S or a combination of different fields with different orientations, such as are described in WO2007129275.

After sufficiently long time, as determined through experimentation, and reversals allowing the formation of substantial amounts of complexes, a sufficient magnetic field MF can be applied by the magnetic field generator 123 in a negative direction away from the sensor surface 110S so that any unbound magnetic particles 115U (unbound to a target component 105T) can be moved away from the sensor surface 110S, such as to a location directly adjacent to the wall opposite from the sensor surface 110S as shown in FIGS. 1G and 1K. Further, the application of the magnetic field MF in the negative direction will also remove any magnetic particles 115B that may be bound to a target component 105T, but that are not specifically complexed to the sensor surface 110S via the target component 105T and second antibody (so called bound yet free-floating target components 105F) as shown in FIG. 1K.

The final magnetic field MF should be of a magnitude low enough so as to not remove any of the previously formed complexes. One example actuation scheme consists of about 1 minute incubation of the test sample 105T causing rehydrating the dry reagents and magnetic particles 115 followed by about 4 minutes of pulsed actuation and about 10 second removal of unbound magnetic particles 115U and any bound yet free-floating target components 105F with the magnetic field generator 123.

Following removal of any unbound magnetic particles 115U and/or any bound yet free-floating target components 105F away from the sensor surface 110S, the amount of the target component 105T (e.g., Troponin I or Troponin T) that is present and complexed to the sensor surface 110S can be determined by any suitable detection method carried out by reader software 126. The assay results may be displayed on a suitable display 128 of the assay device 100. The assay device 100 may further include any suitable user interface 130 for initiating the assay, retrieving assay results, and/or otherwise manipulating data.

For example, referring to FIGS. 1A-1G, detection may be achieved by any suitable detection system 125 that cooperates with the sensor surface 110S in some embodiments. For example, one or more optical sensors can be located in the vicinity of (e.g., under) the quantification window 117, allowing optical detection of magnetic particles 115, which are the complexed to the sensor surface 110S. However, detection and quantification of the concentration of the target component 105T in a test sample 105P with different detection techniques is also possible.

Suitable surface materials and linking chemistries are known to the person of ordinary skill in the art, and are described for instance in “Diagnostic Biosensor Polymers,” by A. M. Usmani and N. Akmal, American Chemical Society, 1994 Symposium Book Series 556, Washington D.C., USA, 1994, in “Protein Architecture, Interfacing Molecular Assemblies and Immobilization Biotechnology,” edited by Y. Lvov and H. Mhwald (Marcel Dekker, New York, 2000), in “The immunoassay Handbook” by David Wild (Nature Publishing Group, London, 2001, ISBN 1-56159-270-6), and in the “Handbook of Biosensors and Electronic Noses. Medicine, Food and the Environment” by Kress-Rogers (ISBN 0-8493-8905-4). Supports for coupling proteins to coated and uncoated plastic and glass supports are also disclosed in Angenendt et al. (2002; Anal Biochem. 309, 253-260).

Detection means suitable for use in the assay methods and assay devices of the present disclosure comprise any detection system 125 capable of detecting a relevant signal such as, but not limited to, an optical signal (reflection, absorption, scattering, fluorescence, chemiluminescence, RAMAN, FTIR, or the like). Active labels that may be coupled to the magnetic particles 115 are known to persons of ordinary skill in the art and include fluorescein dyes, such as 5- (and 6-) carboxy-4′,5′-dichloro-2′,7′-dimethoxy fluorescein, 5-carboxy-2′,4′,5′,7′-tetrachlorofluorescein and 5-carboxyfluorescein, rhodamine dyes such as 5- (and 6-) carboxy rhodamine, 6-carboxytetramethyl rhodamine and 6-carboxyrhodamine X, phthalocyanines such as methyl, nitrosyl, sulphonyl and amino phthalocyanines, azo dyes, azomethines, cyanines and xanthines such as the methyl, nitro, sulphano and amino derivatives, and succinylfluoresceins. Other suitable active labels that can be coupled to the magnetic particles 115 are fluorophores from the group of cyanine dimers and monomers, such as TOTO, YOYO, TO-PRO, Cy3, Cy5, Cy5.5, Cy7, etc., or dyes such as LCRed 705, and can be used as the fluorescent dye.

Depending on the nature of the signal to be detected, the detection system can be an integral part of the sensor surface 110S or can allow the detection of the presence of magnetic labels on the sensor surface 110S. In one example, radioactive labels, such as luminescent or fluorescent labels, are embedded in or otherwise attached to the magnetic particles 115 that are used. Excitation of the fluorescent labels can be done using an irradiation source, such as a focused laser beam or via evanescent field excitation allowing optical detection of such radioactive labels. Detection can be done in any suitable way, such as by using confocal detection or using a high-NA lens. The use of fluorescent labels enables multiplexing by using different fluorophores, which can differ in excitation and/or emission wavelengths, and thus can allow detection of different target components at once.

As mentioned above the detection of magnetic particles 115 complexed at a sensor surface 110S can be measured by any direct or indirect method known in the art. Particular detection methods described herein can be based on optical properties of the magnetic particles 115, such as detection with frustrated total internal reflection (FTIR).

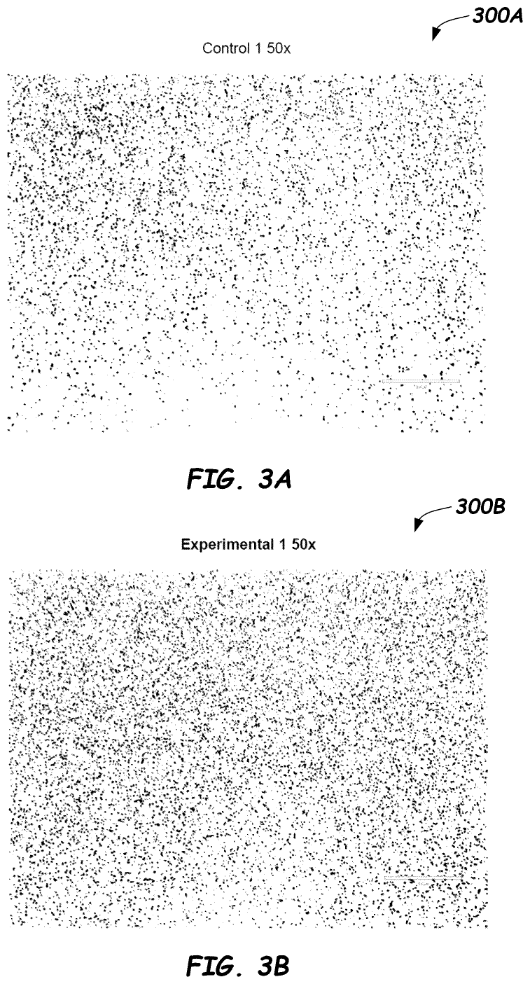

As shown in FIGS. 3A and 3B, particle distribution images 300A, 300B of a sensor surface 110S illustrate experimental results using a particle counter that are shown respectively using a control sample 300A (without application of the pulsed voltage to the test sample 105P with high levels of albumin) in FIG. 3A, and then an experimental sample 300B that included exposing the test sample 105P with high levels of albumin to pulsed voltage as shown in FIG. 3B. Each image is shown at 50× magnification. The images and particle count results illustrate that a large amount of the clumping was reduced over the control (with no voltage pulse applied) based not only on increased particle counts, but also noting that the mean size of the magnetic particles 115 measured was reduced by approximately 6.0% to 7.8% when the pulsed voltage was applied.

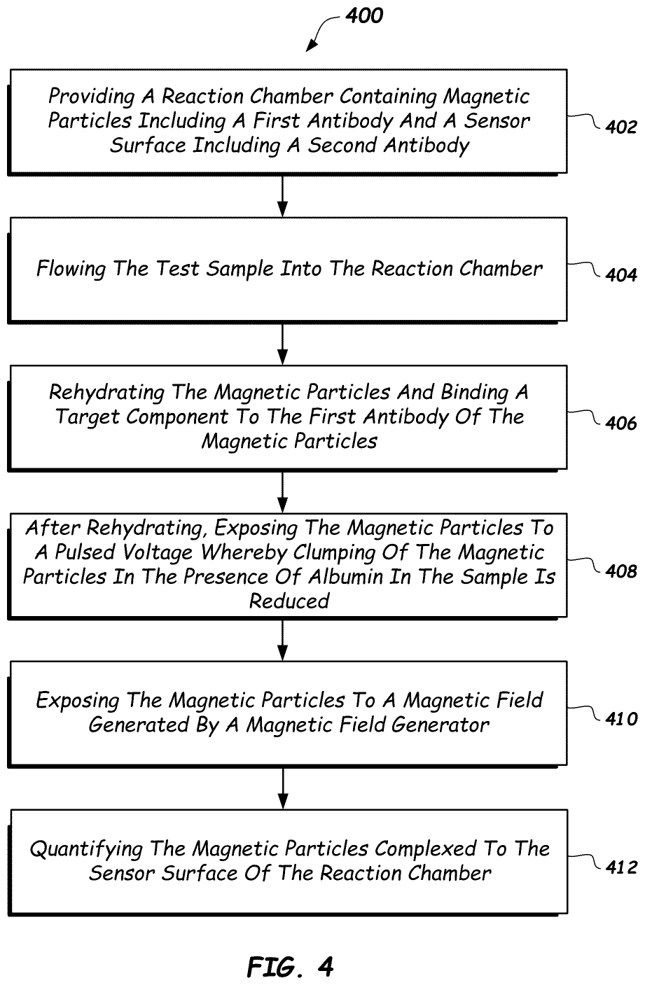

As best shown and described with reference to FIG. 4, a method 400 of quantifying a target component (e.g., Troponin I or Troponin T) in a test sample 105P is provided according to another embodiment of the disclosure. The method 400 comprises, in block 402, providing a reaction chamber (e.g., reaction chamber 110) containing magnetic particles (e.g., magnetic particles 115) including a first antibody (e.g., first antibody 111A) and a sensor surface (e.g., sensor surface 110S) including a second antibody (e.g., second antibody 111B).

The method 400 further comprises, in block 404, flowing the test sample (e.g., test sample 105P) into the reaction chamber (e.g., reaction chamber 110), followed by, in block 406, rehydrating the magnetic particles (e.g., magnetic particles 115) and binding a target component 105T (e.g., Troponin I or Troponin T) to the first antibody 111A of the magnetic particles (e.g., magnetic particles 115).

Additionally, the method 400 further comprises, in block 408, after rehydration in block 406, exposing the magnetic particles (e.g., magnetic particles 115) to a pulsed voltage V whereby clumping (e.g., clumps 115C) of the magnetic particles (e.g., magnetic particles 115) in the presence of albumin in the test sample (e.g., test sample 105P) is reduced.

Following the exposure of the magnetic particles (e.g., magnetic particles 115) to the pulsed voltage V, the magnetic particles (e.g., magnetic particles 115) now including a lesser amount of clumping 115C may be exposed to a magnetic field MF generated by a magnetic field generator (e.g., magnetic field generator 123).

The application of the magnetic field MF provided by the magnetic field generator 123 in a direction towards the sensor surface 110S can provide actuation forces to the magnetic particles 115 carrying the first antibodies 111A during the assay to ensure optimized contact with the sensor surface 110S. In some embodiments, the magnetic field MF can be pulsed, such as by reversing the magnetic flux direction. Magnetic particles 115 can be manipulated in different ways to optimize contact with the immobilized second antibodies 111B provided on the sensor surface 110S.

The reversal of the magnetic field MF can facilitate removal of any magnetic particle 115 from the sensor surface 110S that has no target component 105T bound to the first antibody 111A (e.g., monoclonal anti-troponin I or T antibody), or that may have a target component 105T bound to the first antibody 111A, but is not complexed to the sensor surface 110S. The magnetic particles 115 that have a target component 105T bound to the first antibody 111A and wherein that target component 105T is also bonded to the sensor surface 110S by the second antibody 111B form complexes that remain and thus can be quantified to provide an amount of the target component 105T in the test sample 105P. Several magnetic field reversals may be used to maximize the amount of complexes including the target component on the sensor surface 110S.

In some embodiments, during the step of binding the target component to the first antibody of the magnetic particles, the magnetic particles are exposed to a magnetic field MF from a magnetic field generator to assist in keeping unbound magnetic particles away from the sensor surface 110S. This prevents early binding to the secondary antibody which would otherwise prevent performing a reference signal measurement at the sensor surface.

In some embodiments, following the exposing of the pulsed voltage, the magnetic particles are exposed to a magnetic field MF from a magnetic field generator to assist in moving magnetic particles towards the sensor surface 110S and binding the target component bound to the first antibody to the second antibody at a sensor surface.

In some embodiments, after rehydrating the magnetic particles and before the exposing to the pulsed voltage, the magnetic particles are exposed to a magnetic field MF from a magnetic field generator to assist in binding target components attached to the first antibody to the secondary antibody at the sensor surface, whereby the clumping of the magnetic particles due to the magnetic fields is reduced.

In some embodiments, the pulsed voltage is applied intermittently while exposing the magnetic particles to a magnetic field MF from a magnetic field generator to assist the magnetic particles to capture to the target component via the first antibody, whereby the clumping of the magnetic particles due to the magnetic fields is reduced, and thereby any loss in target component capture efficiency caused by the clumping

In some embodiments, the pulsed voltage is applied intermittently while exposing the magnetic particles to a magnetic field MF from a magnetic field generator to assist the magnetic particles with target components bound to the first antibody, to bind the captured target component to the second antibody at the sensor surface, whereby the clumping of the magnetic particles due to the magnetic fields is reduced, and thereby any loss in binding efficiency of captured target component to the second antibody caused by the clumping. This allows in particular to increase the use of magnetic actuation and enables to apply stronger field strengths and/or to use prolonged periods with actuation since undesired clumping is overcome. The increased use of magnetic fields allows to further speed up the binding processes and thereby enables faster and/or more sensitive assay results.

In some embodiments, the pulsed voltage is applied every 5, 10, 30, 60, and/or 120 seconds.

In some embodiments, the magnetic field MF may be applied substantially simultaneously with one or more pulses of the voltage V to minimize clumping and further aid in maximizing the formation of complexes to the sensor surface 110S. Following application of the magnetic field MF, the results of the assay of the target component 105T can be detected. In particular, in block 412, quantifying the magnetic particles 115 complexed to the sensor surface 110S of the reaction chamber 110 can be accomplished by any of the methods described herein. In some embodiments, the assay cartridge 101 is suitable for optical detection of the magnetic particles 115 complexed to the sensor surface 110S by a detection system 125, such as by FTIR where a range of wavelengths in the infrared region that are absorbed (or reflected) by a material are measured.

FTIR involves using, as shown in FIG. 1A-1G, a source 125L, such as an illumination source (e.g., an infrared (IR) source projected onto the quantification window 117 and thus onto the magnetic particles 115 complexed to the sensor surface 110S. In some embodiments, the projection can be provided along the quantification window through the use of one or more prisms. The test sample's absorbance of the infrared light energy at various wavelengths can be measured by receiver 125R and recorded. This data may be used to determine a quantity of magnetic particles 115 complexed to the sensor surface 110S, which can be substantially directly proportional to the quantity of the target component 105T present in the test sample 105P. Other suitable optical quantification methods may be used.

The following is a list of non-limiting illustrative embodiments disclosed herein:

1. A method of quantifying a target component in a test sample, comprising:
providing a reaction chamber containing magnetic particles including a first antibody and a sensor surface including a second antibody;
flowing the test sample into the reaction chamber;
rehydrating the magnetic particles and binding a target component to the first antibody of the magnetic particles; and
after rehydration, exposing the magnetic particles to a pulsed voltage whereby clumping of the magnetic particles in the presence of albumin in the test sample is reduced.
   2. The method of the first illustrative embodiment, wherein the target component is Troponin I (cTnI) or Troponin T (cTnT).
   3. The method of any one of the illustrative embodiments 1 to 2, wherein the magnetic particles are provided with a monoclonal anti-Troponin antibody comprising the first antibody, and/or wherein the first antibody is a monoclonal anti-Troponin antibody.
   4. The method of any one of the illustrative embodiments 1 to 3, comprising a sensor surface, wherein the sensor surface is located proximate to a quantification window of the reaction chamber and the sensor surface is provided with a polyclonal anti-Troponin antibody comprising a second antibody.
   5. The method of any one of the illustrative embodiments 1 to 4, wherein the pulsed voltage comprises a magnitude greater than 150 V.
   6. The method of any one of the illustrative embodiments 1 to 5, wherein the pulsed voltage comprises a magnitude greater than 300 V.
   7. The method of any one of the illustrative embodiments 1 to 6, wherein the exposing of the pulsed voltage comprises providing at least two voltage pulses to break up the clumping of the magnetic particles.
   8. The method of the illustrative embodiment 7, wherein each of the at least two voltage pulses have a duration of greater than 2 milliseconds.
   9. The method of any one of the illustrative embodiments 1 to 8, wherein during the step of binding the target component to the first antibody of the magnetic particles, the magnetic particles are exposed to a magnetic field MF from a magnetic field generator to assist in keeping unbound magnetic particles away from the sensor surface 110S.
   10. The method of any one of the illustrative embodiments 1 to 9, wherein following the exposing of the pulsed voltage, the magnetic particles are exposed to a magnetic field MF from a magnetic field generator to assist in moving magnetic particles towards the sensor surface 110S and binding the target component bound to the first antibody to the second antibody at a sensor surface.
   11. The method of any one of the illustrative embodiments 1 to 10, wherein following the exposing of the pulsed voltage, the magnetic particles are exposed to a magnetic field MF from a magnetic field generator to assist in moving unbound magnetic particles away from the sensor surface 110S.
   12. The method of any one of the illustrative embodiments 1 to 11, wherein after rehydrating the magnetic particles and before the exposing to the pulsed voltage, the magnetic particles are exposed to a magnetic field MF from a magnetic field generator to assist in binding target components attached to the first antibody to the secondary antibody at the sensor surface, whereby the clumping of the magnetic particles due to the magnetic fields is reduced.
   13. The method of any one of the illustrative embodiments 1 to 12, wherein the pulsed voltage is applied intermittently while exposing the magnetic particles to a magnetic field MF from a magnetic field generator to assist the magnetic particles to capture to the target component via the first antibody.
   14. The method of any one of the illustrative embodiments 1 to 13, wherein the pulsed voltage is applied intermittently while exposing the magnetic particles to a magnetic field MF from a magnetic field generator to assist the magnetic particles with target components bound to the first antibody, to bind the captured target component to the second antibody at the sensor surface.
   15. The method of any one of the illustrative embodiments 1 to 14, wherein the pulsed voltage is applied every 5, 10, 30, 60, and/or 120 seconds.
   16. The method of any one of the illustrative embodiments 1 to 15, comprising an inlet channel configured to flow the test sample to the reaction chamber, wherein the inlet channel is a capillary channel.
   17. The method of the illustrative embodiment 1 to 16, comprising a filter configured to allow substantially only test sample to pass through to the reaction chamber.
   18. The method of any one of the illustrative embodiments 1 to 17, wherein the albumin is present in a sample from which the test sample is derived in a quantity greater than 5.4 g/dL in whole blood.
   19. The method of the illustrative embodiment 18, wherein the albumin is present in a in a quantity greater than 10 g/dL in whole blood.
   20. The method of the illustrative embodiment 18, wherein the albumin is present in a quantity greater than 100 g/dL in whole blood.
   21. An assay cartridge, comprising:
a cartridge body including a sample inlet adapted to receive a sample and a reaction chamber coupled to the sample inlet;
magnetic particles contained in the reaction chamber comprising a first antibody with a specific affinity towards a target component;
electrodes provided at a location configured to be in contact with a rehydrated test sample containing the magnetic particles in the reaction chamber; and
a sensor surface of the reaction chamber comprising a second antibody with a specific affinity towards the target component.
   22. The assay cartridge of the illustrative embodiment 21, wherein the target component comprises Troponin I or Troponin T.
   23. The assay cartridge of any one of the illustrative embodiments 21 to 22, further comprising a first electrode positioned on a first side of the reaction chamber and a second electrode positioned on a second side of the reaction chamber.
   24. The assay cartridge of any one of the illustrative embodiments 21 to 23, further comprising a filter configured to retain red blood cells and allow the test sample to pass through the filter into the reaction chamber.
   25. The assay cartridge of any one of the illustrative embodiments 21 to 24, wherein the magnetic particles are provided with a monoclonal antibody comprising the first antibody.
   26. The assay cartridge of any one of the illustrative embodiments 21 to 25, further wherein a sensor surface proximate a quantification window of the reaction chamber is provided with a polyclonal antibody comprising the second antibody.
   27. An assay device, comprising:
a device body including a cartridge receiver;
an assay cartridge received in the cartridge receiver, the assay cartridge comprising a cartridge body including an inlet channel adapted to receive a test sample and a reaction chamber therein, magnetic particles having a first antibody with a specific affinity towards a target component provided in the reaction chamber, and electrodes provided at locations configured to be in contact with a rehydrated test sample containing the magnetic particles in the reaction chamber; and
a voltage pulse generator coupled to the electrodes and configured to provide one or more voltage pulses to the electrodes.

While this disclosure is susceptible to various modifications and alternative forms, specific method, assay cartridge, and assay device embodiments have been shown by way of example in the drawings and are described in detail herein. It should be understood, however, that the particular method, assay cartridges, and assay devices disclosed herein are not intended to limit the disclosure or the claims.

What is claimed is:

1. A method of quantifying a target component in a test sample, comprising:
providing a reaction chamber containing magnetic particles including a first antibody and a sensor surface including a second antibody;
flowing the test sample into the reaction chamber;
rehydrating the magnetic particles and binding a target component to the first antibody of the magnetic particles; and
after rehydration, exposing the magnetic particles to a pulsed voltage whereby clumping of the magnetic particles in a presence of albumin in the test sample is reduced.

2. The method of claim 1, wherein the target component is Troponin I or Troponin T.

3. The method of claim 1, wherein the first antibody is a monoclonal anti-Troponin antibody.

4. The method of claim 1, comprising a sensor surface, wherein the sensor surface is located proximate to a quantification window of the reaction chamber and the sensor surface is provided with a polyclonal anti-Troponin antibody comprising a second antibody.

5. The method of claim 1, wherein the pulsed voltage comprises a magnitude greater than 150 V.

6. The method of claim 1, wherein the pulsed voltage comprises a magnitude greater than 300 V.

7. The method of claim 1, wherein the exposing of the pulsed voltage comprises providing at least two voltage pulses to break up the clumping of the magnetic particles.

8. The method of claim 7, wherein each of the at least two voltage pulses have a duration of greater than 2 milliseconds.

9. The method of claim 1, wherein during the step of binding the target component to the first antibody of the magnetic particles, the magnetic particles are exposed to a magnetic field MF from a magnetic field generator to assist in keeping unbound magnetic particles away from the sensor surface 110S.

10. The method of claim 1, wherein following the exposing of the pulsed voltage, the magnetic particles are exposed to a magnetic field MF from a magnetic field generator to assist in moving magnetic particles towards the sensor surface 110S and binding the target component bound to the first antibody to the second antibody at a sensor surface.

11. The method of claim 1, wherein following the exposing of the pulsed voltage, the magnetic particles are exposed to a magnetic field MF from a magnetic field generator to assist in moving unbound magnetic particles away from the sensor surface 110S.

12. The method of claim 1, wherein after rehydrating the magnetic particles and before the exposing to the pulsed voltage, the magnetic particles are exposed to a magnetic field MF from a magnetic field generator to assist in binding target components attached to the first antibody to the secondary antibody at the sensor surface, whereby the clumping of the magnetic particles due to the magnetic fields is reduced.

13. The method of claim 1, wherein the pulsed voltage is applied intermittently while exposing the magnetic particles to a magnetic field MF from a magnetic field generator to assist the magnetic particles to capture the target component via the first antibody.

14. The method of claim 1, wherein the pulsed voltage is applied intermittently while exposing the magnetic particles to a magnetic field MF from a magnetic field generator to assist the magnetic particles with target components bound to the first antibody, to bind the captured target component to the second antibody at the sensor surface.

15. The method of claim 14, wherein the pulsed voltage is applied every 5, 10, 30, 60, and/or 120 seconds.

16. The method of claim 1, comprising an inlet channel configured to flow the test sample to the reaction chamber, wherein the inlet channel is a capillary channel.

17. The method of claim 16, comprising a filter configured to allow substantially only test sample to pass through to the reaction chamber.

18. The method of claim 1, wherein the albumin is present in a sample from which the test sample is derived in a quantity greater than 5.4 g/dL in whole blood.

19. The method of claim 18, wherein the albumin is present in a in a quantity greater than 10 g/dL in whole blood.

20. The method of claim 18, wherein the albumin is present in a quantity greater than 100 g/dL in whole blood.

21. An assay cartridge, comprising:
a cartridge body including a sample inlet adapted to receive a sample and a reaction chamber coupled to the sample inlet;
magnetic particles contained in the reaction chamber comprising a first antibody with a specific affinity towards a target component;
electrodes provided at a location configured to be in contact with a rehydrated test sample containing the magnetic particles in the reaction chamber; and
a sensor surface of the reaction chamber comprising a second antibody with a specific affinity towards the target component.

22. The assay cartridge of claim 21, wherein the target component comprises Troponin I or Troponin T.

23. The assay cartridge of claim 21, further comprising a first electrode positioned on a first side of the reaction chamber and a second electrode positioned on a second side of the reaction chamber.

24. The assay cartridge of claim 21, further comprising a filter configured to retain red blood cells and allow the test sample to pass through the filter into the reaction chamber.

25. The assay cartridge of claim 21, wherein the magnetic particles are provided with a monoclonal antibody comprising the first antibody.

26. The assay cartridge of claim 21, further wherein a sensor surface proximate a quantification window of the reaction chamber is provided with a polyclonal antibody comprising the second antibody.

27. An assay device, comprising:
a device body including a cartridge receiver;
an assay cartridge received in the cartridge receiver, the assay cartridge comprising a cartridge body including an inlet channel adapted to receive a test sample and a reaction chamber therein, magnetic particles having a first antibody with a specific affinity towards a target component provided in the reaction chamber, and electrodes provided at locations configured to be in contact with a rehydrated test sample containing the magnetic particles in the reaction chamber; and
a voltage pulse generator coupled to the electrodes and configured to provide one or more voltage pulses to the electrodes.
